(19) United States
(12) Patent Application Publication
(10) Pub. No.: US 2025/0108149 A1
(43) Pub. Date: Apr. 3, 2025

(54) HYALURONIC ACID HYDROGELS AND METHODS THEREOF

(71) Applicant: The Governing Council of the University of Toronto, Toronto (CA)

(72) Inventors: Gayatri Prakash, Toronto (CA); Frank Gu, Toronto (CA); Shengyan Liu, Cambridge, MA (US)

(73) Assignee: The Governing Council of the University of Toronto, Toronto (CA)

(21) Appl. No.: 18/901,943

(22) Filed: Sep. 30, 2024

Related U.S. Application Data
(60) Provisional application No. 63/541,256, filed on Sep. 28, 2023.

(51) Int. Cl. A61L 27/52

(52) U.S. Cl. CPC A61L 27/52

(57) ABSTRACT

The present application relates to hyaluronic acid (HA) hydrogels comprising divalent cations and hyaluronic acid molecules cross-lined with the divalent cations, and uses thereof. The present application also relates to methods of preparing the HA hydrogels of the present application.

### CROSS-REFERENCE TO RELATED APPLICATIONS

The present application claims the benefit of priority from co-pending U.S. provisional patent application No. 63/541,256 filed on Sep. 28, 2023, the contents of which are incorporated herein by reference in their entirety.

### FIELD

The present application relates to hyaluronic acid hydrogels (HA hydrogels) comprising divalent cations and methods of preparation thereof. The present application also relates to biomaterials comprising the HA hydrogels of the present application.

### INTRODUCTION

Hyaluronic acid (HA) is a ubiquitous biopolymer with natural presence in the body including in ocular tissues, between joints and in skin. The application of HA and HA-based hydrogels in the biomedical industry is widely explored due to the polymer's biocompatibility, biodegradability and hydrophilicity. Common biomedical applications of HA hydrogels include their use as viscosupplements for joints, lubricants for the ocular surface, scaffolds for tissue engineering, surgical implants, and drug delivery matrices. HA alone, while capable of forming highly viscoelastic solutions due to chain entanglements, is unable to form stable hydrogels without incorporating a crosslinker. Common strategies to prepare HA hydrogels use crosslinking of HA chains through covalent interactions. However, the irreversibility of these crosslinks can limit application in drug delivery by hindering responsiveness to external stimuli, and the rigidity of these matrices result in biomaterials with poor mechanical properties (i.e. brittleness) that insufficiently capture the dynamic nature of biological tissues. While dynamic covalent and non-covalent crosslinking strategies are being explored, these strategies commonly necessitate modification of the HA backbone, which may present issues with biocompatibility as well as clearance.

Accordingly, there is a need to develop alternative non-covalent cross-linking strategies for HA to prepare HA hydrogels.

### SUMMARY

Divalent cations are previously known to decrease viscosity of HA. However, it has been surprisingly shown herein that divalent cations (such as Mg²⁺, Zn²⁺, Mn²⁺, Fe²⁺, Co²⁺, Pd²⁺, and Ni²⁺) can be used to crosslink HA chains to form HA hydrogels through non-covalent interactions. This is particularly unexpected since, unlike Cu²⁺, these metals, especially the alkaline earth metal Mg²⁺, are not as well-known to undergo electronic transitions to stabilize their interactions with a ligand (i.e. the Jahn Teller distortion).

Accordingly, in one aspect, the present application includes a method of preparing a hyaluronic acid (HA) hydrogel comprising
combining an aqueous HA solution and an aqueous cation solution comprising divalent cation to obtain a mixture, or
adding a solid divalent cation salt to an aqueous HA solution to obtain a mixture,
wherein
   either (a) the aqueous HA solution has a pH of about 10 to about 14, or
   (b) the method further comprises adjusting the pH of the mixture to about 10 to about 14; and
wherein the divalent cation is selected from Mg²⁺, Zn²⁺, Mn²⁺, Fe²⁺, Co²⁺, Pd²⁺, Ni²⁺, and combinations thereof.

In another aspect, the present application includes a method of preparing a hyaluronic acid (HA) hydrogel comprising
combining an aqueous HA solution and an aqueous cation solution comprising divalent cation to obtain a mixture, or
adding a solid divalent cation salt to an aqueous HA solution to obtain a mixture,
wherein
   either (a) the aqueous HA solution has a pH of about 10 to about 14, or
   (b) the method further comprises adjusting the pH of the mixture to about 10 to about 14; and
wherein the divalent cation has an ionic radius of about 100 pm or less than 100 pm.

In another aspect, the present application includes a hyaluronic acid (HA) hydrogel comprising divalent cations and HA molecules crosslinked non-covalently with the divalent cations, wherein the divalent cations are selected from Mg²⁺, Zn²⁺, Mn²⁺, Fe²⁺, Co²⁺, Pd²⁺, Ni²⁺, and combinations thereof.

In another aspect, the present application includes a hyaluronic acid (HA) hydrogel prepared by the method of the present application.

In another aspect, the present application includes a use of a HA hydrogel of the present application in the preparation of a biomaterial.

In another aspect, the present application includes a biomaterial comprising a HA hydrogel of the present application.

In another aspect, the present application includes a use of a HA hydrogel of the present application in tissue engineering.

In another aspect, the present application includes a use of a HA hydrogel of the present application in 3D printing, optionally as a 3D printing ink.

In another aspect, the present application includes a use of a HA hydrogel of the present application in drug delivery, for example in cancer therapy.

In another aspect, the present application includes use of a HA hydrogel of the present application in water treatment.

In another aspect, the present application includes use of a HA hydrogel of the present application in wearable electronics.

In another aspect, the present application includes use of a HA hydrogel of the present application in antimicrobial treatment.

In another aspect, the present application includes a HA hydrogel of the present application for use in the preparation of a biomaterial.

In another aspect, the present application includes a HA hydrogel of the present application for use in tissue engineering.

In another aspect, the present application includes a HA hydrogel of the present application for use in 3D printing. In another aspect, the present application includes a HA hydrogel of the present application for use in the preparation of a 3D printing ink.

In another aspect, the present application includes a HA hydrogel of the present application for use in drug delivery, for example in cancer therapy.

In another aspect, the present application includes a HA hydrogel of the present application for use in water treatment.

In another aspect, the present application includes a HA hydrogel of the present application for use in wearable electronics.

In another aspect, the present application includes a HA hydrogel of the present application for use in antimicrobial treatment.

### DRAWINGS

The embodiments of the disclosure will now be described in greater detail with reference to the attached drawings in which:

FIG. 1 shows a heat map showing the propensity of HA hydrogel bead formation in the presence of various exemplary divalent cations including Cu²⁺, Zn²⁺, Fe²⁺, Ca²⁺, Mg²⁺, and Mn²⁺.

FIG. 2 shows HA hydrogel formation with further cations. Panel A shows select photographs of hydrogel beads in the gelation solution (pH 12 HA, 64 mM ion). Cross-hatched squares indicate that no hydrogels are formed. Black squares indicate transient hydrogel formation that quickly ended as ion-HA precipitates. For all ions that were able to form ion-HA hydrogels, Panel B shows the conditions under which hydrogel beads formed. The relative success of hydrogel formation was rated uniquely for each ion. Within each condition the first box refers to hydrogels formed on the day of preparation, and the second refers to those hydrogels the following day. The Shannon radii of the various ions tested are shown in Panel C, where squares indicate hydrogel formation, an “x” indicates no hydrogel formation, and circles indicate that the ion has not been tested. Shannon radii values are obtained from [2].

FIG. 3 shows HA hydrogel formation in the presence of exemplary MgCl₂ in panel A and ZnCl₂ in panel B.

FIG. 4 shows hydrogel formation with exemplary ion salt ZnCl₂ with 1% w/v HA and different concentration of divalent cation. Panel A shows images of the hydrogels in inverted tubes. Panel B shows a graph showing variation of viscosity with shear rate in comparison with divalent ion-free (i.e. no added ion, or added Na⁺), and non-gelling (Ca²⁺) ion-containing controls. Curves with error bars represent an average of n=2-4, with error bars representing standard deviation. The variation of the storage (G′) and loss (G″) moduli with frequency over two days across the ZnCl₂ concentration range tested is shown in Panel C. Gel formation is indicated by G′>G″.

FIG. 5 shows heat maps showing the effect of HA concentration and molar ratio to divalent cation on hydrogel formation using exemplary Zn²⁺ (Panel A) and exemplary Mg²⁺ (Panel B).

FIG. 6 shows hydrogels formed with various exemplary cations at different concentrations by titration with NaOH. Panel A shows pictures of the resulting exemplary hydrogels being lifted out of the solution vials. Panel B shows the LVE, G′ and G″ and panel C the variation of viscosity with shear rate.

FIG. 7 shows in Panels A to D graphs of variation of tan δ with angular frequency measured by frequency sweep experiments for solutions of HA with CuCl₂, ZnCl₂, MgCl₂, and CaCl₂ respectively treated with base as described in Example 7. Representative resulting exemplary hydrogels being lifted with a pipet tip are shown as insets. Panel E shows graph of variation of tan δ with angular frequency measured by frequency sweep experiments for solutions of HA with exemplary MgCl₂ treated with different bases, with pictures of select hydrogels in the inset.

FIG. 8 shows pictures of the vials containing solutions of HA, CaCO₃ and glucono-δ-lactone as comparative examples.

FIG. 9 shows the storage modulus and tan δ of the resulting mixture formed using various divalent cations and HA without addition of NaOH (Panel A) or with addition of NaOH (Panel B). Solid lines indicate storage modulus and dotted lines indicate tan δ.

FIG. 10 shows pictures of HA hydrogels formed with further cations by titration with NaOH as described in Example 10.

FIG. 11 shows in panels A and D pictures of HA hydrogels formed with Mg²⁺ while varying the concentration of MgCl₂ (panel A) or NaOH (panel D) within the hydrogels, respectively. Panels B and E show graphs of the variation of storage modulus (top) and tan δ (bottom) with varying angular frequency while varying the concentration of MgCl₂ (panel B) or NaOH (panel E) in the hydrogels, respectively. Panels C and F show graphs of storage and loss moduli of the HA hydrogels at different angular frequencies with varying concentrations of MgCl₂ (panel C) or NaOH (panel F), respectively and demonstrate the plateauing effect.

FIG. 12 shows in panels A and C pictures of HA hydrogels formed with Mg²⁺ while varying the concentration (panel A) or molecular weight (panel C) of HA in the hydrogels, respectively. Panels B and D show graphs of the variation of storage modulus (top) and tan δ (bottom) with varying angular frequency while varying the concentration (panel B) or molecular weight (panel D) of HA in the hydrogels, respectively.

FIG. 13 shows in panel A the rheological profile of a HA hydrogel crosslinked with MgCl₂ under repetitive cycles of high and low stress. In panel B, the stability and ion-exchange mediated reversibility of an example HA hydrogel with Mg²⁺ as crosslinker is shown.

FIG. 14 shows pictures of the breakdown of a Mg-HA hydrogel in EDTA.

Other features and advantages of the present application will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DESCRIPTION OF VARIOUS EMBODIMENTS

### I. Definitions

Unless otherwise indicated, the definitions and embodiments described in this and other sections are intended to be applicable to all embodiments and aspects of the present disclosure herein described for which they are suitable as would be understood by a person skilled in the art.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the disclosure exist as individual salts and hydrates, as well as a combination of, for example, a solvate of a salt of a compound of the disclosure.

As used in the present disclosure, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this disclosure and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art. The term “reversible” as used herein in the context of HA hydrogel refers to the ability of a hydrogel to reverse the gelation process, e.g. to disintegrate, and revert to free HA molecules in solution.

The term “hyaluronic acid (HA)” as used herein includes both the acid and conjugate base forms (hyaluronate).

### II. Methods and Uses of the Disclosure

Accordingly, in one aspect, the present application includes a method of preparing a hyaluronic acid (HA) hydrogel comprising
combining an aqueous HA solution and an aqueous cation solution comprising divalent cation to obtain a mixture, or
adding a solid divalent cation salt to an aqueous HA solution to obtain a mixture,
wherein
   either (a) the aqueous HA solution has a pH of about 10 to about 14, or
   (b) the method further comprises adjusting the pH of the mixture to about 10 to about 14; and wherein the divalent cation is selected from Mg²⁺, Zn²⁺, Mn²⁺, Fe²⁺, Co²⁺, Pd²⁺, Ni²⁺, and combinations thereof.

In another aspect, the present application includes a method of preparing a hyaluronic acid (HA) hydrogel comprising
combining an aqueous HA solution and an aqueous cation solution comprising divalent cation to obtain a mixture, or
adding a solid divalent cation salt to an aqueous HA solution to obtain a mixture,
wherein
   either (a) the aqueous HA solution has a pH of about 10 to about 14, or
   (b) the method further comprises adjusting the pH of the mixture to about 10 to about 14; and
wherein the divalent cation has an ionic radius of about 100 pm or less than 100 pm.

In some embodiments, the divalent cation excludes Cu²⁺ and Ca²⁺. In some embodiments, the divalent cation is selected from Mg²⁺, Zn²⁺, Mn²⁺, Fe²⁺, Co²⁺, Pd²⁺, Ni²⁺, and combinations thereof.

In some embodiments, in (a) the aqueous HA solution has a pH of about 10 to about 14, and the combining comprises adding the aqueous HA solution to the aqueous cation solution in a dropwise manner.

In some embodiments, in (b) the method further comprises adjusting the pH of the mixture to about 10 to about 14, and the divalent cation is selected from Mg²⁺, Zn²⁺, Mn²⁺, Fe²⁺, Co²⁺, Pd²⁺, Ni²⁺, and combinations thereof. For example, in (b) the method further comprises adjusting the pH of the mixture to about 10 to about 14, and the divalent cation is Fe²⁺.

Without wishing to be bound by theory, it can be appreciated that when the aqueous HA solution is added to the aqueous cation solution, for example in a dropwise manner, under conditions (a), the local concentration and pH near where the is added changes first and relatively more than elsewhere in the remaining resulting mixture. Accordingly, it is observed that gelation occurs first where the drop of HA solution is added. It can then be appreciated that in some embodiments, when the combining is carried out without stirring, the HA hydrogel is in bead form.

Without wishing to be bound by theory, when the combining of the aqueous HA solution with the aqueous cation solution or solid divalent cation salt is carried out under conditions (a) with simultaneous stirring, the local concentration of the added cation does not remain different from elsewhere in the resulting mixture, and the gelation occurs in an amorphous manner. Therefore, in some embodiments, the combining is carried out with simultaneous mixing (e.g. stirring, vortexing), and the HA hydrogel is amorphous.

Without wishing to be bound by theory, when adjusting the pH of the mixture of aqueous HA solution and divalent cation under conditions (b) with simultaneous stirring, the local pH of the mixture where the pH adjusting agent is dropped changes first and relatively more than elsewhere in the remaining mixture, until stirring eventually disperses the pH adjusting agent across the entire mixture. Gelation thus occurs locally first and eventually across the mixture in an amorphous manner resulting in homogenously or in homogenously crosslinked hydrogels.

In some embodiments, the solid divalent cation salt, such as a halide salt, for example a chloride salt, is added as a solid to the aqueous solution in an amount to provide a desired concentration of cation in the resulting mixture. In some embodiments, the addition is carried out with simultaneous stirring. In some embodiments the solid divalent cation salt is MgCl₂.

In some embodiments, the aqueous HA solution has a pH of about 11 to about 13.5, or the aqueous HA solution has a pH of about 12 to about 13.5.

In some embodiments, in (b) the method comprises adjusting the pH of the mixture to about 10 to about 13.5, and the combining of the aqueous HA solution and the aqueous cation solution or the solid divalent cation salt and the adjusting of the pH are carried out with simultaneous mixing (e.g. stirring, vortexing), and wherein the HA hydrogel is amorphous.

In some embodiments, the adjusting of the pH of the mixture is carried out with a strong base. For example, the strong base is a hydroxide base, Tris base or mixtures thereof. In some embodiments, the strong base is an alkali hydroxide. Suitable alkali hydroxide includes NaOH, KOH, and combinations thereof. In some embodiments, the strong base is selected from NaOH, KOH, and combinations thereof.

In some embodiments, the base is added as an aqueous solution wherein the concentration of the base in the solution is about 0.25 M to about 4 M, about 0.35 M to about 3 M, about 0.5 M to about 2 M or about 1 M.

In some embodiments, the pH of the mixture is adjusted to about 11 to about 13.5, to about 12 to about 13, or to about 12.5 to about 13.

In some embodiments, the strong base is present in the mixture at a concentration of about 10 mM to about 400 mM, about 15 mM to about 350 mM, about 15 mM to about 300 mM, or about 15 mM to about 280 mM.

In some embodiments, the mixture has a concentration of HA of at least about 0.125% w/v, at least about 0.2% w/v, at least about 0.5% w/v, or at least about 1% w/v. In some embodiments, the mixture has a concentration of HA of less than about 20% w/v, less than about 15% w/w, less than about 10% w/v, less than about 7% w/v, less than about 5% w/v, or less than about 3% w/v. In some embodiments, the mixture has a concentration of HA of about 0.1% w/v to about 4.5% w/v, about 0.1% w/v to about 4.0% w/v, about 0.1% w/v to about 3.5% w/v, about 0.1% w/v to about 3.0% w/v, about 0.125% w/v to about 2.5% w/v, about 0.25% w/v to about 2.5% w/v, about 0.75% w/v to about 2% w/v, about 0.5% w/v to about 2% w/v, or about 1% w/v.

In some embodiments, the divalent cation is present in the mixture at a concentration of about 3 mM to about 100 mM, about 10 mM to about 80 mM, about 15 mM to about 70 mM, about 20 mM to about 60 mM, about 25 mM to about 50 mM, about 25 mM to about 45 mM or about 30 mM.

In some embodiments, the divalent cation is Mg²⁺ or Zn²⁺.

In some embodiments, the divalent cation is Mg²⁺.

In some embodiments, the concentration of the HA in the mixture is about 0.5% w/v to about 1.5% w/v, and wherein the HA and the Mg²⁺ are present in the mixture at a molar ratio of HA to Mg²⁺ of about 0.3 to about 2.5.

In some embodiments, the concentration of the HA in the mixture is about 0.25% w/v to about 0.5% w/v, and wherein the HA and the Mg²⁺ are present in the mixture at a molar ratio of HA to Mg²⁺ of about 0.3 to about 0.8.

In some embodiments, the divalent cation is Zn²⁺.

In some embodiments, the concentration of the HA in the mixture is about 0.5% w/v to about 1.5% w/v, and wherein the HA and the Zn²⁺ are present in the mixture at a molar ratio of HA to Zn²⁺ of about 0.3 to about 2.5.

In some embodiments, the concentration of the HA in the mixture is about 0.25% w/v to about 0.5% w/v, and wherein the HA and the Zn²⁺ are present in the mixture at a molar ratio of HA to Zn²⁺ of about 0.3 to about 0.8.

In some embodiments, the divalent cation is in the form of its chloride salt, nitrate salt, acetate salt, sulfate salt and mixtures thereof. In some embodiments, the divalent cation is in the form of its chloride salt and/or nitrate salt.

The term hyaluronic acid (HA) as used herein includes both the acid and conjugate base forms (hyaluronate). Therefore, in some embodiments, the aqueous HA solution comprises HA as a hyaluronate salt or as an acid, or a mixture thereof. For example, the HA is sodium and/or potassium hyaluronate. In some embodiments, the aqueous HA solution comprises HA as in acid form.

In some embodiments, the HA has a molecular weight of about 300 kDa to about 1000 kDa, about 300 kDa to about 950 kDa, about 400 kDa to about 950 kDa, about 500 kDa to about 950 kDa, about 600 kDa to about 950 kDa, about 700 kDa to about 950 kDa, or about 800 kDa to about 900 kDa.

In some embodiments, the HA has a molecular weight of about 200 kDa to about 1700 kDa, about 250 kDa to about 1600 kDa, about 300 kDa to about 1500 kDa, about 400 kDa to about 1400 kDa, about 500 kDa to about 1300 kDa, about 600 kDa to about 1200 kDa, about 700 kDa to about 1100 kDa, about 800 kDa to about 1000 kDa or about 800 kDa to about 900 kDa.

In another aspect, the present application includes a use of a HA hydrogel of the present application in the preparation of a biomaterial.

In another aspect, the present application includes a use of a HA hydrogel of the present application in tissue engineering.

In another aspect, the present application includes a use of a HA hydrogel of the present application in 3D printing, optionally as a 3D printing ink.

In another aspect, the present application includes a use of a HA hydrogel of the present application in drug delivery, for example in cancer therapy. In some embodiments, the drug delivery is ionic environment-responsive drug delivery.

In another aspect, the present application includes use of a HA hydrogel of the present application in water treatment.

In another aspect, the present application includes use of a HA hydrogel of the present application in wearable electronics.

In another aspect, the present application includes use of a HA hydrogel of the present application in antimicrobial treatment.

In another aspect, the present application includes a HA hydrogel of the present application for use in the preparation of a biomaterial.

In another aspect, the present application includes a HA hydrogel of the present application for use in tissue engineering.

In another aspect, the present application includes a HA hydrogel of the present application for use in 3D printing. In another aspect, the present application includes a HA hydrogel of the present application for use in the preparation of a 3D printing ink.

In another aspect, the present application includes a HA hydrogel of the present application for use in drug delivery, for example in cancer therapy. In some embodiments, the drug delivery is ionic environment-responsive drug delivery.

In another aspect, the present application includes a HA hydrogel of the present application for use in water treatment.

In another aspect, the present application includes a HA hydrogel of the present application for use in wearable electronics.

In another aspect, the present application includes a HA hydrogel of the present application for use in antimicrobial treatment.

In another aspect, the present application includes a method of preparing a biomaterial comprising combining a HA hydrogel of the present application with other components of the biomaterial under conditions to prepare the biomaterial. In some embodiments, the biomaterial is tissue and the method is a method of preparing or engineering tissue.

In another aspect, the present application includes a method of 3D printing comprising printing an ink comprising a HA hydrogel of the present application onto a substrate using a 3D printer.

In another aspect, the present application includes a method of delivering a drug to a subject in need thereof comprising administering a HA hydrogel of the present application to the subject. In some embodiments, the drug is an anticancer drug and the subject has cancer or is at risk of having cancer. In some embodiments, the drug is an antimicrobial drug and the subject has a microbial infection or is at risk of having a microbial infection. In some embodiments, the method comprises ionic environment-responsive drug delivery.

In another aspect, the present application includes a method of treating water comprising adding use of a HA hydrogel of the present application in water treatment.

In another aspect, the present application includes a method of preparing a wearable electronic comprising combining a HA hydrogel of the present application with other components of the wearable electronic under conditions to prepare the wearable electronic.

### II. HA Hydrogel and Biomaterials of the Disclosure

In another aspect, the present application includes a hyaluronic acid (HA) hydrogel comprising divalent cations and HA molecules crosslinked non-covalently with the divalent cations, wherein the divalent cations are selected from Mg²⁺, Zn²⁺, Mn²⁺, Fe²⁺, Co²⁺, Pd²⁺, Ni²⁺, and combinations thereof.

In another aspect, the present application includes a hyaluronic acid (HA) hydrogel prepared by the method of the present application.

In some embodiments, the HA hydrogel comprises divalent cations and HA molecules cross-linked non-covalently with the divalent cations.

In some embodiments, the divalent cation is Zn²⁺, and the HA hydrogel has a pH of about 6 to 8, or about 7.

In some embodiments, the divalent cation is Mg²⁺, and the HA hydrogel has a pH of about 9 to about 11, about 10.

In some embodiments, the HA hydrogel is biocompatible.

In some embodiments, the HA hydrogel is a self-healing hydrogel. Self-healing refers to spontaneous formation of new bonds when old bonds are broken within the hydrogel.

In some embodiments, the HA hydrogel is injectable.

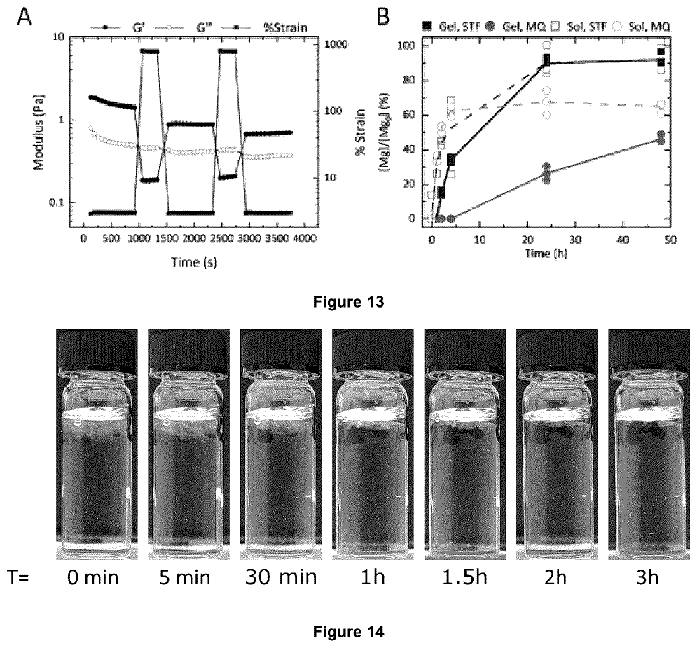

Without wishing to be bound by theory, since the divalent cations and the HA molecules are not covalently attached, their interactions can be disrupted by additions of external chemicals. For instance, when a HA hydrogel of the present application is placed in high concentration of salt (e.g. NaCl), or ion chelator (e.g. EDTA) the sodium cation may eventually replace the divalent cations in the HA hydrogel, or the chelator chelate the divalent cations in the HA hydrogel, respectively, leading to the reversal of gelation back to a solution of HA (as shown in FIGS. 13 and 14). The same may occur when the HA hydrogel of the present application is placed in water for exceedingly long periods of time, where the divalent cations may slowly diffuse out of the HA hydrogel causing the hydrogel to eventually disintegrate. It can be appreciated this process may depend on the concentration of divalent cations in the HA hydrogel, the ionic strength of the salt solution used to contact the HA hydrogel, the relative affinity of the chelator used for the divalent cation compared to its affinity with HA, and the length of time the HA hydrogel is exposed to water. As such, in some embodiments, the HA hydrogel is reversible. In some embodiments, the reversibility of the HA hydrogel into HA solution is ion-exchange mediated or chelation mediated.

In some embodiments, the HA hydrogel is flexible. In some embodiments, the HA hydrogel is a viscoelastic solid. In some embodiments, the HA hydrogel is a semi-solid. In some embodiments, the HA hydrogel has a storage modulus (G′) between 10⁻¹ Pa and 10² Pa. In some embodiments, the HA hydrogel has a loss modulus (G″) less than the storage modulus at least at the angular frequency range below 10⁻¹ rad/s while within the linear viscoelasticity regime. In some embodiments, the HA hydrogel has a tan δ of less than 1 at least at the angular frequency range below 10⁻¹ rad/s while within the linear viscoelasticity regime. In some embodiments, a tan δ between 1 and 0.1 at least at the angular frequency range below 10⁻¹ rad/s while within the linear viscoelasticity regime.

In another aspect, the present application includes a biomaterial comprising the HA hydrogel of the present application.

In some embodiments, the biomaterial is selected from viscosupplement, biolubricant, drug delivery matrix, surgical implant, tissue engineering scaffold, and artificial tissue.

### EXAMPLES

The following non-limiting examples are illustrative of the present disclosure.

### General Methods

Hyaluronic acid (˜800 kDa-900 kDa) was obtained from either HTL Biotech or Lifecore Biomedical. No apparent differences in hydrogel formation were observed between the two sources. Magnesium(II) Chloride (MgCl₂), Manganese(II) Chloride (MnCl₂), Cobalt(II) Chloride (CoCl₂), Nickel(II) Chloride (NiCl₂), Calcium(II) Chloride (CaCl₂)), Zinc(II) Chloride (ZnCl₂), Copper(II) Chloride (CuCl₂), Iron(II) Chloride (FeCl₂), Strontium(II) Chloride (SrCl₂), Barium(II) Chloride (BaCl₂), Silver Nitrate (AgNO₃), Sodium Chloride (NaCl), Potassium Chloride (KCl), and Palladium(II) Nitrate (Pd(NO₃)₂) were dissolved to prepare the various solutions of cations. Milli-Q® water (18.2MΩ-cm) was used in the preparation of all aqueous solutions. Various hydrates or anhydrous forms of the salts were used.

Unless otherwise stated, titration to a pH above pH 7 was carried out using 1 M NaOH, and titration to a pH below pH 6-7 was carried out using 1 M HCl. A Sure-Flow™ pH electrode was used to determine the pH of solutions after titration.

Unless otherwise stated, rheological experiments were generally carried out using a DHR-3 rheometer equipped with a 40 mm aluminum parallel plate with a gap height of 100 um.

### Example 1 Preliminary Screen of Divalent Ions for Ionic Crosslinkinq of HA

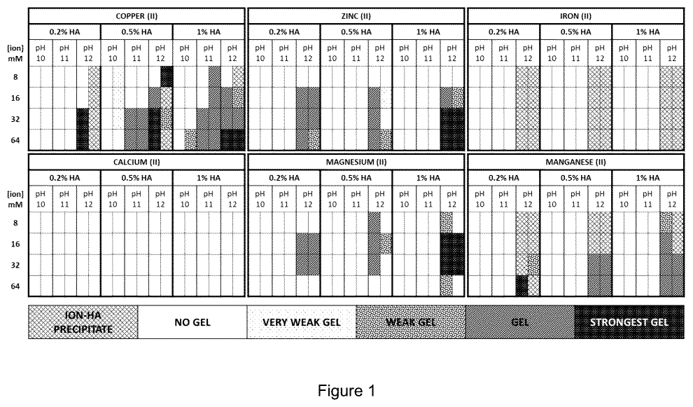

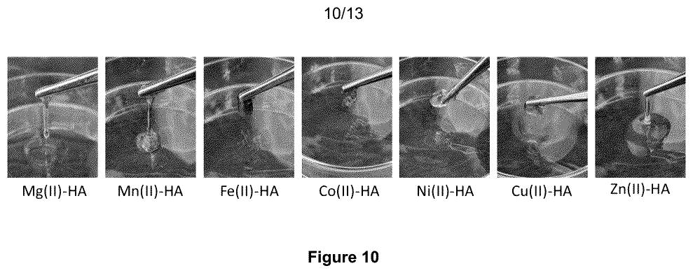

800 kDa-900 kDa HA at various concentrations (0.2% w/v, 0.5% w/v, or 1% w/v) was titrated to pH 10, 11 or 12 prior to dropwise addition to solutions of divalent ions (chloride salts of the divalent ions were used) under no agitation. The vials were visually screened for formation of hydrogel-like substances or hydrogel beads, and rated according to the following scale: ion-HA precipitate, no gel, very weak gel, weak gel, gel, and strong gel. FIG. 1 is a heatmap showing the propensity for hyaluronic acid (HA) hydrogel bead formation in the presence of divalent cations. The two boxes under a particular condition indicate the stability of the hydrogels over the course of 18 hours. Hydrogel formation was observed in the presence of Cu(II) as well as Zn(II), Mg(II) and Mn(II). Cu(II) formed the most stable hydrogels, followed by Zn(II), Mg(II) and finally Mn(II). Interestingly, Fe(II) rapidly formed precipitates, which may be the insoluble Fe(OH)₂ salt, or tightly associated Fe-HA complexes. It was later shown herein below that Fe(II) did form hydrogel when HA solution and divalent ion solution were combined and the pH of the mixture adjusted to high pH of about 10 to about 14. (See Example 10 and FIG. 10) Ca(II) formed no precipitates or hydrogels. In general, it was noted that higher concentrations of hyaluronic acid, ions as well as alkaline pH promoted hydrogel bead formation.

### Example 2 Extended Screen of Divalent Ions for Ionic Cross-Linking of HA

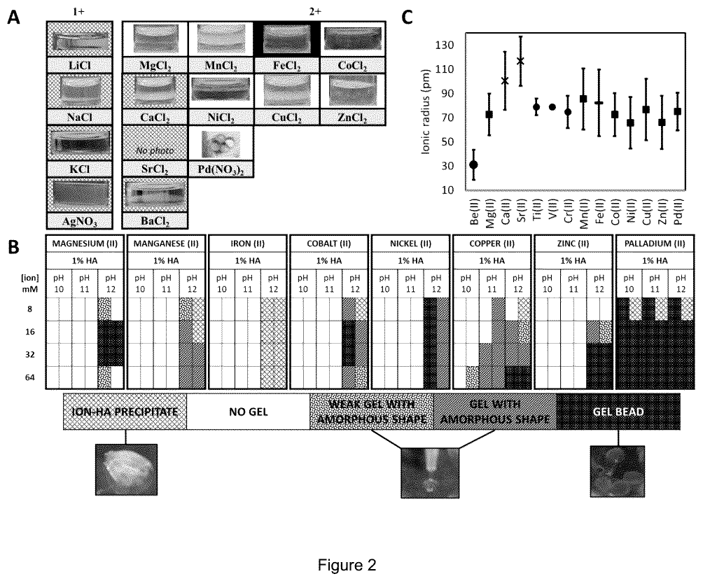

800 kDa-900 kDa HA at a 1% concentration was titrated to pH 10, 11 or 12 prior to dropwise addition to solutions of divalent ions under no agitation. Vials were visually screened for hydrogel bead formation and monitored over 18 h. FIGS. 2A-B expand findings from FIG. 1, and demonstrate the applicability of the methods of the present application to other divalent ions to form hydrogel beads. Further to the ions used in Example 1, Co(II), Ni(II), and Pd(II) were also able to cross-link HA using the method of Example 1. FIG. 2C demonstrates that the ability of an ion to cross-link HA depends on the ionic properties of the ion: Without wishing to be bound by theory, it was observed that smaller ions with a smaller ionic radius tend to more readily cross-link HA.

### Example 3 Hydrogel Bead Formation

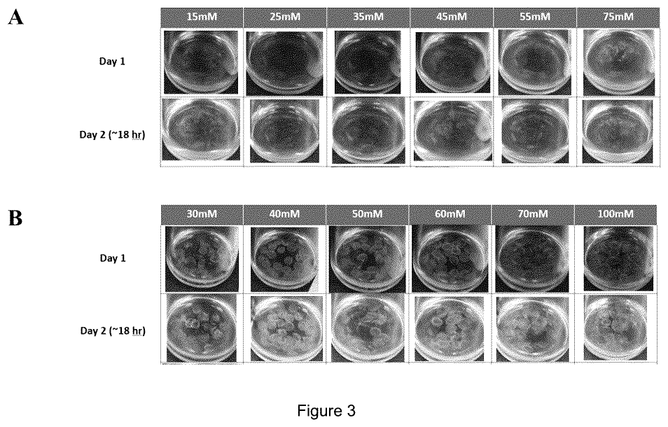

1% w/v 800 kDa-900 kDa hyaluronic acid was titrated to a pH of 12.5 prior to addition to MgCl₂ or ZnCl₂ solutions, dropwise under no agitation, at the various concentrations listed. The stability of the beads was monitored visually over 18 h. Pictures of the hydrogel beads are shown in FIG. 3. Without wishing to be bound by theory, the stability and morphology of the beads depended on the concentrations of the ions. Zinc-HA beads appeared more stable than magnesium-HA beads.

### Example 4 Cation Concentration

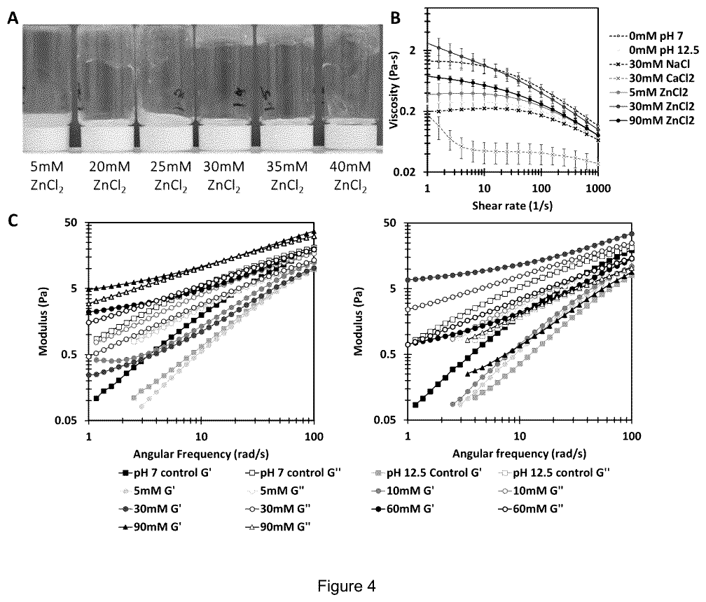

Ion solutions (pH 3.2) were mixed with HA (pH 12.5) to yield ion-HA mixtures containing 1% w/v HA (800 kDa-900 kDa) at various ion concentrations shown. Gelation was verified by tube inversion method. Example images of the hydrogels are shown in FIG. 4A. Viscosity of the gels is shown in FIG. 4B. Gels made with various ZnCl₂ concentrations were measured for their variation of storage and loss moduli with frequency over two days, which are shown in FIG. 4C.

Optimal hydrogel formation for a 1% w/v HA solution occurred at 30 mM ZnCl₂. The optimal gels formed under these conditions were found to be near physiological pH (pH 7.1±0.2). FIG. 4B shows that HA gels formed with ZnCl₂ using the method as described above in this Example showed increases in viscosity, with Bingham fluid-like yield stress compared to divalent ion-free (i.e. no added ions, or addition of equimolar concentration of NaCl) and non-gelling ion-containing (CaCl₂)) controls. FIG. 4C shows the variation of the storage (G′) and loss (G″) moduli with frequency over time and across ZnCl₂ concentrations. In accordance with visual observations, optimal hydrogel formation for a 1% w/v HA solution occurred in the presence of 30 mM ZnCl₂, where G′>G″ over the frequency range, and occurred on the day after the ZnCl₂ and HA were mixed. Weak gel formation on the day of ZnCl₂ and HA mixing was also observed with higher concentrations of ZnCl₂, where G′>G″ for some frequencies.

### Example 5 HA Concentration and Ratio of HA to Cation

HA at various concentrations (1% w/v, 0.5% w/v, 0.25% w/v, 0.125% w/v) was titrated to a pH of 12.5 with 1 M NaOH prior to mixing with ion solutions at the HA monomer:ion molar ratios of 5:1, 2.5:1, 0.8:1, 0.4:1, or 0.3:1. The HA solution was added to the ion solution dropwise (HA->ion), or the ion solutions were added to the HA solutions dropwise (ion->HA) under 500 rpm stirring. The solutions were stirred for 20 minutes, and gel formation was monitored over 18-24 h. Zn-HA hydrogel formation was evaluated using the tube inversion test, and Mg-HA hydrogel formation was evaluated based on the ability to lift a gel out of solution. Squares with no shading indicate no gel formation. Dotted squares indicate a transient gel with low stability. Dark squares indicate successful gel formation.

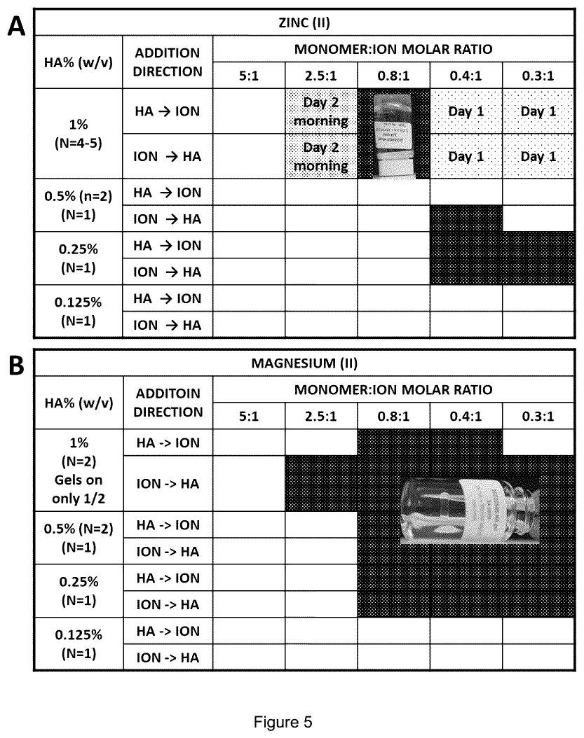

FIG. 5 shows the effects of the crosslinking ion concentration and HA concentration on gel formation. Gel formation was observed for both Zn²⁺ and Mg²⁺ at different HA concentrations and HA monomer to cation molar ratios.

### Example 6 Hydroqel Formation by Base Titration

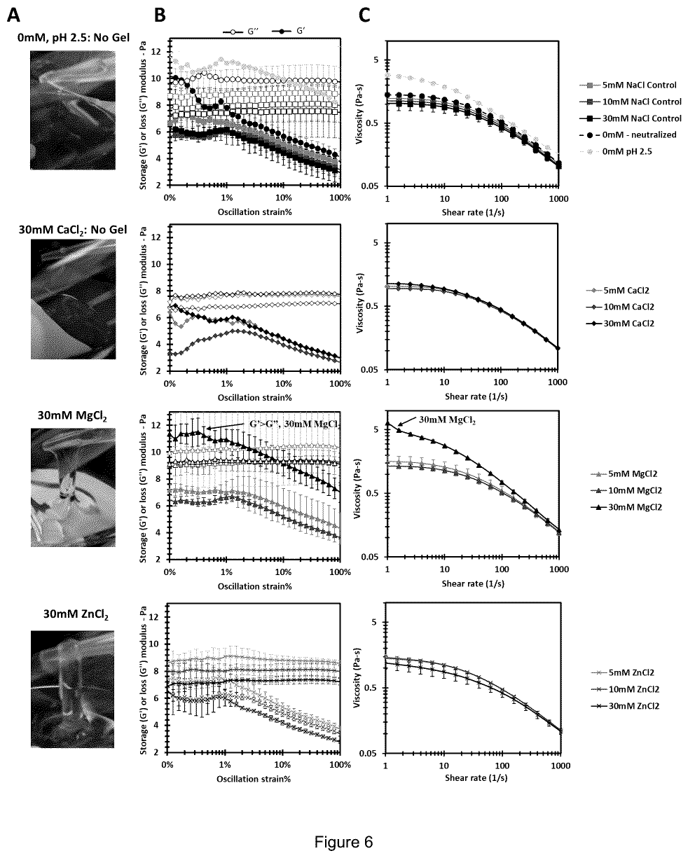

MgCl₂, ZnCl₂, CaCl₂), NaCl, or milliQ™ water (pH 2.5-2.6) were mixed with HA (pH 2.5-2.6) to yield 1% (w/v) HA solutions at various ion concentrations (0 mM, 5 mM, 10 mM, or 30 mM), and slowly titrated with 1M NaOH under rapid stirring until the solution containing the formed hydrogels reached a pH of 5-8. While not wishing to be limited by theory, it is believed that the addition of 1 M NaOH locally increases the pH of the solution to >pH 12, resulting in localized hydrogel formation. Representative resulting hydrogels were able to be lifted with a pipet tip as shown in FIG. 6A. CaCl₂), NaCl and milliQ™ water at pH 2.5 were used as negative controls. Strain-sweep experiments were conducted to determine the linear viscoelasticity regime (LVE), G′ and G″ and flow-sweep experiments were conducted to determine the variation of viscosity with shear rate. Results are shown in FIG. 6B and FIG. 6C.

Hydrogel formation in the presence of Zn²⁺ and Mg²⁺ was qualitatively confirmed by lifting the resulting hydrogel out of solution. Hydrogel formation in the presence of Mg²⁺ was also confirmed using rotational rheology, with G′>G″ in a strain-sweep experiment, as shown in FIG. 4B. ZnCl₂ hydrogels showed lower stability compared to MgCl₂. While photographs of only 30 mM hydrogels are shown in FIG. 6A, hydrogel formation occurred at ion concentrations of 5 mM and 10 mM as well in presence of both Mg²⁺ and Zn²⁺, with lower stability. Moreover, the hydrogels formed under these conditions were found to be between pH 4-8, as determined by a pH electrode.

### Example 7 Effect of pH and Base

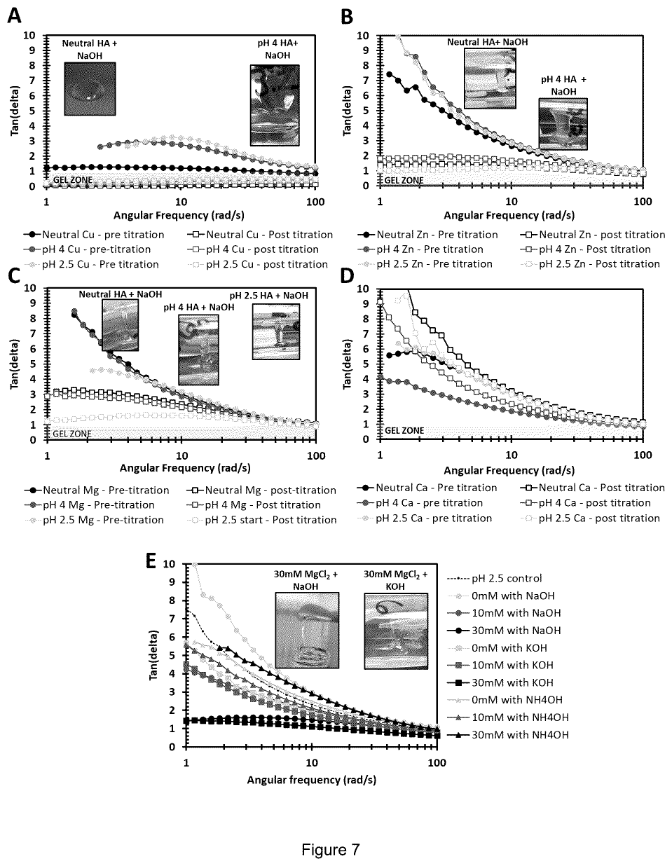

CuCl₂, ZnCl₂, MgCl₂, or CaCl₂ was mixed with HA to yield 6 mL ion-HA mixtures containing 30 mM ion and 1% (w/v) HA, and pre-titrated to pH 2.6 or 4 using 1M HCl, or left as is. The solutions were then slowly titrated with 60 μL of 1 M NaOH under 300 rpm stirring. Frequency sweep experiments were conducted by rotational rheology and the variation of tan δ with angular frequency is shown for each sample before (pre-titration) and after (post-titration) NaOH addition in FIG. 7A to D.

MgCl₂ and HA were premixed to yield 6 mL ion-HA mixtures containing 1% HA (w/v) and 0, 10, or 30 mM MgCl₂, and pre-titrated to pH 2.6 using 1 M HCl. The solutions were then slowly titrated with 60 uL of 1 M base (NaOH, KOH, NH₄OH, or Tris base) under 300 rpm stirring, and stirred for 45 s. The variation of tan δ with angular frequency is shown in FIG. 7E for each sample after base addition. Lower tan δ values indicate entanglement or crosslinking.

FIG. 7 demonstrates that pre-titration of an HA solution to low pH (pH 2.6) is not required for hydrogel formation. Instead, the addition of a base (e.g. NaOH or KOH) that is capable of increasing the pH to a high enough level initiated hydrogel formation. FIG. 7A-C show decreases in tan δ, which indicates increased entanglement or crosslinking, when NaOH is added to an HA solution containing Cu(II), Zn(II) and Mg(II), regardless of the initial pH to which the solutions were titrated. In contrast, increases in tan δ were observed under the same conditions with Ca(II). FIG. 7E further shows that NaOH and KOH can be used interchangeably to increase the pH of the solutions to enable crosslinking (decrease in tan δ in presence of the base and ion), while NH₄OH (increase in tan δ) and Tris Base (data not shown) alone cannot be used in this manner. NH₄OH and Tris worked with Zn(II).

### Example 8 Comparative Example

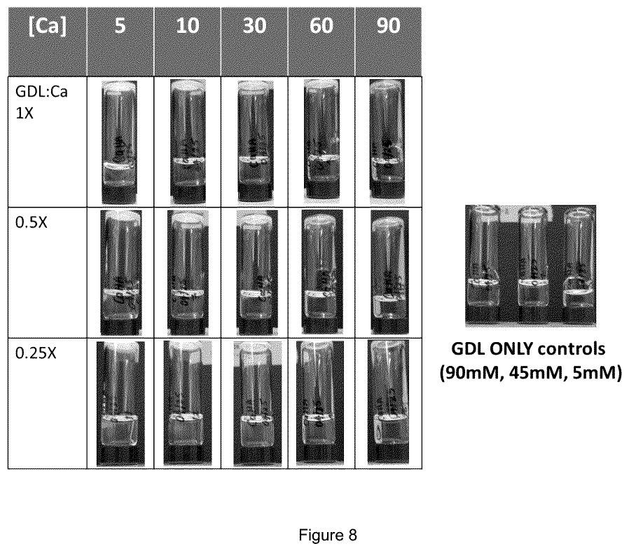

A comparative example was done by mixing HA with an insoluble Ca(II)-salt (e.g. CaCO₃) and glucono-δ-lactone (GDL). It was observed that no hydrogel formed and Ca(II) could not crosslink HA. The reaction conditions (decreasing pH) were not conducive to HA hydrogel formation. In this comparative example, 1% (w/v) hyaluronic acid in DI water was mixed with varying concentrations of CaCO₃, to which GDL was added GDL:Ca ratios of 1×, 0.5× and 0.25×. The mixtures were vortexed for 20-30 s, and the solutions were monitored over 18 h. Hydrogel formation was assessed using the vial flip method. After 18 h, no hydrogel was observed. FIG. 8 shows that no hydrogels formed under any of the conditions tested.

### Example 9 Oscillatory Rheoloqy of the HA Hydrogels

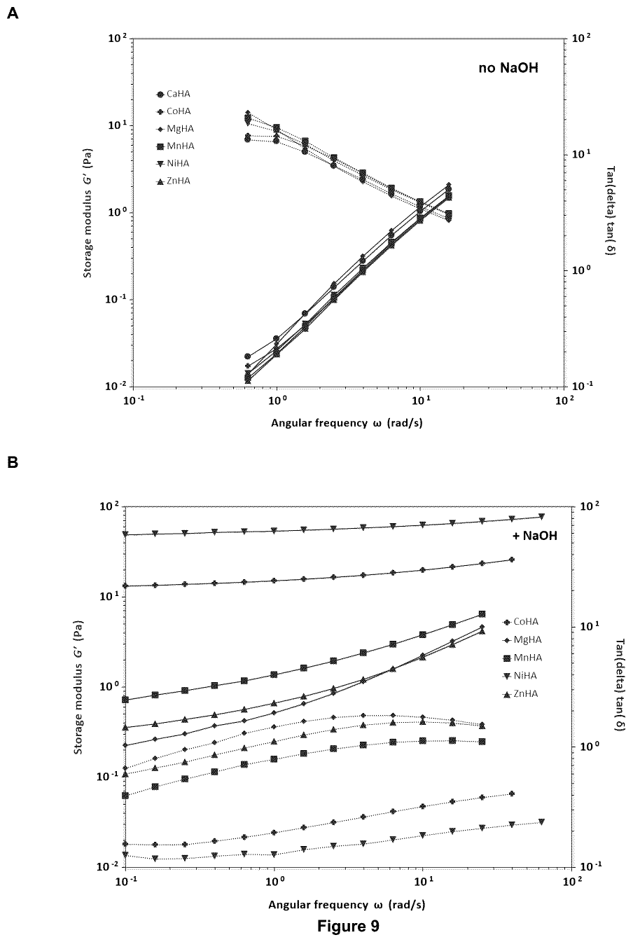

800 kDa-900 kDa sodium hyaluronate (2% w/v) was mixed with 60 mM CaCl₂, MgCl₂, MnCl₂, CoCl₂, NiCl₂ or ZnCl₂ at a 1:1 volume ratio to yield HA-ion solutions at a final concentration of 1% w/v HA and 30 mM ion. The solutions (1.1 mL) were loaded and spread onto the rheometer, followed by the addition of 78.6 μL of either MilliQ™ water or 1 M NaOH. The top plate (60 mm 1 degree cone plate) of the rheometer was immediately lowered to the experimental gap height while rotating at an angular velocity of 0.1 rad/s to mix the two solutions. Rheological tests were performed immediately after. Frequency sweep experiments were conducted at a 1% strain, which was verified to be within the linear viscoelasticity regimes of all solutions and hydrogels. A solvent trap was used to limit water evaporation over the course of the experiment. FIG. 9A shows the variation of the storage modulus (G′) and tan δ with frequency in the absence of NaOH. FIG. 9B shows the same for hydrogels that result with addition of NaOH. Solid lines indicate storage modulus, and dotted lines indicate tan δ. A higher storage modulus indicates a stronger hydrogel, and lower tan δ indicates greater elasticity.

FIG. 9 shows that for all ions except Ca²⁺, the viscosity of which was too low to accurately determine the viscoelastic properties of the resulting solution, the addition of NaOH to the ion-HA solution resulted in increases in storage modulus and decreases in tan δ when compared to the non-NaOH containing controls. The tan δ for all ion-HA hydrogels lay below 1 at low frequencies, confirming hydrogel formation. The G′ and tan δ also varied with the ion used for crosslinking, where across the first row transition metals (i.e. Mn(II), Co(II), Ni(II), Zn(II)) smaller ions generally resulted in stronger and more elastic hydrogels closely following the Irving Williams series. The viscoelastic properties of the Mg-HA hydrogels were similar to those of the Zn-HA hydrogels.

### Example 10 Extended Demonstration of Hydrogel Formation by Base Titration

Solutions of 800 kDa-900 kDa sodium hyaluronate (HA) were mixed with MgCl₂, MnCl₂, FeCl₂, CoCl₂, NiCl₂, CuCl₂ or ZnCl₂ solutions to yield 0.7 mL solutions containing 1% HA w/v and 30 mM of the respective ion. 50 μL of 1 M NaOH was subsequently added to the tube and immediately vortex mixed for about 10 seconds. FIG. 10 shows representative images of the resulting hydrogels. Interestingly, the method of this Example results in stable hydrogel formation for all the ions specified, including Fe(II). Hydrogel strength generally followed as Zn(II)-HA<Mg(II)-HA<Mn(II)-HA<Fe(II)-HA<Co(II)-HA<Ni(II)-HA<Cu(II)-HA.

### Example 11 Demonstration of Altering Rheological Properties of Hydrogels with Varying Cation or Base Concentrations

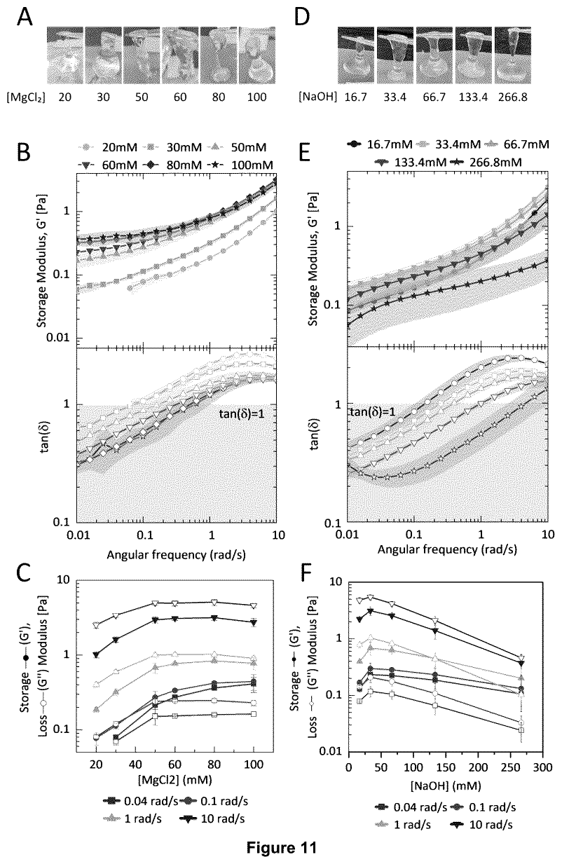

Solutions of 800 kDa-900 kDa sodium hyaluronate (HA) were mixed with MgCl₂ to yield 1.1 mL solutions containing 1% w/v HA and ion concentration ranging from 20 mM to 100 mM. 78.6 uL of 1M NaOH was subsequently added to the tube and immediately vortex mixed for about 10-30 seconds. The samples were tested by oscillatory shear rheology using a 60 mm cone plate equipped with a solvent trap under 5% strain at 25 C. This strain % was confirmed to be within the linear viscoelasticity regime of all hydrogels tested. FIG. 11A shows representative images of the resulting hydrogels. FIG. 11B shows graphs of the variation of storage modulus (top) or tan δ (bottom) with varying angular frequency. Increasing the concentration of divalent cation was found to decrease the tan δ further below 1, indicating an increase in the solid-like properties of the resulting hydrogels. Increasing the divalent cation concentration was also shown to increase the storage modulus of the hydrogels till a plateau of no further gains in gel strength was observed. FIG. 11C better shows this plateauing effect with increasing divalent cation concentration.

To demonstrate the effect of varying the concentration of NaOH in the HA hydrogels prepared with MgCl₂, solutions of 800 kDa-900 kDa sodium hyaluronate were mixed with solutions of MgCl₂ to yield 1.1 mL solutions containing 1% w/v HA and 30 mM MgCl₂. 78.6 uL of NaOH was added to the tube and vortex mixed immediately for 10-30 s to yield HA hydrogels containing final NaOH concentrations ranging from 16.7 mM to 266.8 mM. The samples were tested by oscillatory shear rheology using a 60 mm cone plate equipped with a solvent trap under 5% strain at 25 C. This strain % was confirmed to be within the linear viscoelasticity regime of all hydrogels tested. FIG. 11D shows representative images of the resulting hydrogels. FIG. 11E shows graphs of the variation of storage modulus (top) or tan δ (bottom) with varying angular frequency. Increasing the concentration of NaOH was found to decrease the tan δ further below 1 especially at lower angular frequencies, indicating an increase in the solid-like properties of the resulting hydrogels. Similar to the effects observed with changing the concentration of divalent cation crosslinker within the hydrogels, increasing the NaOH concentration was shown to also increase the storage modulus of the hydrogels till a plateau or eventual decrease in storage modulus at excess concentrations of NaOH. FIG. 11F better shows this plateauing effect with increasing NaOH concentration.

### Example 12 Demonstration of Altering Rheological Properties of Hydrogels with Varying HA Concentration, or Molecular Weight

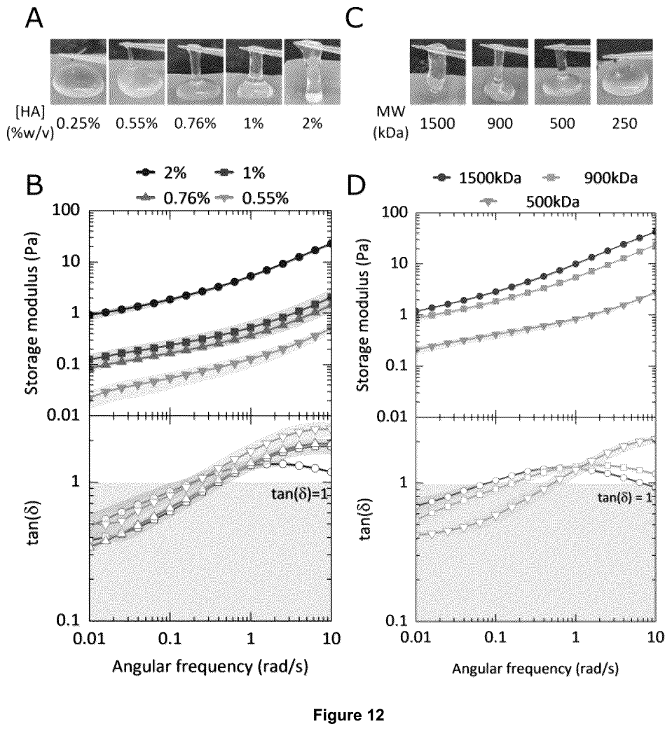

Solutions of 800 kDa-900 kDa sodium hyaluronate (HA) were mixed with MgCl₂ to yield 1.1 mL solutions containing HA concentrations ranging from 0.1% w/v to 2% w/v and 30 mM MgCl₂. 78.6 uL of NaOH was subsequently added to the tube such that the molar ratio between NaOH and HA monomer was maintained at 1.2, and immediately vortex mixed for about 10-30 seconds. The samples were tested by oscillatory shear rheology using a 60 mm cone plate equipped with a solvent trap under 5% strain at 25 C. This strain % was confirmed to be within the linear viscoelasticity regime of all hydrogels tested. FIG. 12A shows representative images of the resulting hydrogels depicting an increase in hydrogel strength with increasing HA concentration. FIG. 12B shows graphs of the variation of storage modulus (top) or tan δ (bottom) with varying angular frequency. Increasing the concentration of divalent cation showed an increase the storage modulus, while maintaining the tan δ relatively constant. This indicated an increase in gel strength with increasing HA concentration, while the solid-like nature of the material was maintained constant.

To demonstrate the effect of varying the molecular weight of HA within the HA hydrogels prepared with MgCl₂, solutions of sodium hyaluronate of molecular weights ranging from 250 kDa to 1500 kDa were mixed with solutions of MgCl₂ to yield 1.1 mL solutions containing 2% w/v HA and 30 mM MgCl₂. 78.6 uL of 2M NaOH was added to the tube and vortex mixed immediately for 10-30 s. The samples were tested by oscillatory shear rheology using a 60 mm cone plate equipped with a solvent trap under 5% strain at 25 C. This strain % was confirmed to be within the linear viscoelasticity regime of all hydrogels tested. FIG. 12C shows representative images of the resulting hydrogels, and depicts qualitatively an increase in hydrogel strength with increasing HA molecular weight. FIG. 12D shows graphs of the variation of storage modulus (top) or tan δ (bottom) with varying angular frequency. Increasing the molecular weight of HA was found to increase the storage modulus, but also surprisingly increase the tan δ. This indicated that although lower molecular weights of HA resulted in weaker hydrogels, the extent of crosslinking within the hydrogels likely increased.

### Example 13 Demonstration of the Potential Self-Healing Ability and Ion Exchange-Mediated Breakdown of the HA Hydrogels

Solutions of 800 kDa-900 kDa sodium hyaluronate (HA) were mixed with MgCl₂ to yield 1.1 mL solutions containing 1% w/v HA and 50 mM MgCl₂. 78.6 uL of 1M NaOH was subsequently added to the tube and immediately vortex mixed. The resulting hydrogel was subjected to repetitive stress-strain measurements to test self-healing behaviour using oscillatory shear rheology. The HA hydrogel was subjected to a low strain (3%) at an angular frequency of 0.1 rad/s for 900 s and subsequently a high strain (800%) at the same angular frequency for 300 s in a cyclic manner a total of two times, before allowing the hydrogel to recover its properties under the low strain condition for 900 s. The resulting rheological profile is depicted in FIG. 13A wherein the hydrogels consistently demonstrated solid-like character under the low strain condition with G′>G″, while demonstrating liquid-like character (G″>G′) under the high strain conditions that break up the hydrogel structure. This modulus reversal is characteristic of materials that demonstrate self-healing behaviour, and demonstrates the potential for injectability.

HA hydrogels (1% HA w/v, 30 mM MgCl₂, 66.7 mM NaOH) and solutions (1% HA w/v, 30 mM MgCl₂) prepared according to the above-described procedure were placed in dialysis cassettes and submerged in water or simulated tear fluid to evaluate the stability and ion-exchange mediated breakdown of the materials. The Mg²⁺ concentration was tested at regular intervals within the dialysate using inductively coupled plasma optical emission spectroscopy (ICP-OES), where an increase in the concentration of Mg²⁺ within the dialysate served as a proxy for hydrogel breakdown. FIG. 13B shows the percentage of Mg²⁺ released into the dialysate over 48 h across the four conditions tested. The hydrogels were found to retain Mg²⁺ more than the solution counterparts in both water and simulated tear fluid indicating superior stability of the hydrogels in the challenge solutions compared to the solutions that result without NaOH. The hydrogels were also found to release more Mg²⁺ over 48 h in the ion-containing simulated tear fluid compared to the ion-free water conditions. This, combined with the EDTA-mediated breakdown of similarly prepared hydrogels depicted qualitatively in FIG. 14 demonstrated that the HA hydrogels prepared using the methods described herein can be reversed into free HA solution with ion exchange or chelation mediated displacement of the divalent cation crosslinker.

### Example 14: Formation of hyaluronic acid hydrogels at higher hyaluronic acid concentration

Solid ion chloride (ion=Mg²⁺, Ca²⁺, Mn²⁺, Fe²⁺, Co²⁺, Ni²⁺ or Zn²⁺) was added to solutions of 800 kDa-900 kDa sodium hyaluronate (HA) to yield 110 uL solutions containing 4% w/v HA and 200 mM ion chloride. The solutions were vortex mixed till homogenous and the salt was completely dissolved through the HA solution. 8 μL of 4M NaOH was subsequently added to the solution and vortex mixed for 10-30 s. When the ion chloride used was one of Mg²⁺, Mn²⁺, Fe²⁺. Co²⁺, Ni²⁺ or Zn²⁺, this procedure resulted in hydrogels. In contrast when Ca²⁺ was used, the resulting mixture remained a solution, and had decreased viscosity compared to the original Ca(II)-HA mixture without NaOH.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### FULL CITATIONS OF REFERENCES REFERRED TO IN THE PRESENT APPLICATION

[1]. Schmut and H. Hofmann, “Preparation of gels from hyaluronate solutions,” Graefe's Arch Clin Exp Ophthalmol, vol. 218, no. 6, pp. 311-314, June 1982, doi: 10.1007/BF02150446.
[2]. “Database of Ionic Radii.” R. D. Shannon, “Revised Effective Ionic Radii and Systematic Studies of Interatomic Distances in Halides and Chalcogenides”, Acta Cryst. A32 751-767 (1976).

1. A method of preparing a hyaluronic acid (HA) hydrogel comprising
combining an aqueous HA solution and an aqueous cation solution comprising divalent cation to obtain a mixture, or
adding a solid divalent cation salt to an aqueous HA solution to obtain a mixture,
wherein
either (a) the aqueous HA solution has a pH of about 10 to about 14 or
(b) the method further comprises adjusting the pH of the mixture to about 10 to about 14; and
wherein the divalent cation is selected from Mg²⁺, Zn²⁺, Mn²⁺, Fe²⁺, Co²⁺, Pd²⁺, Ni²⁺, and combinations thereof.

2. The method of claim 1, wherein in (a) the aqueous HA solution has a pH of about 10 to about 13.5, and the combining comprises adding the aqueous HA solution to the aqueous cation solution in a dropwise manner, or the combining comprises adding the solid divalent cation salt to an aqueous HA solution.

3. The method of claim 2, wherein the combining comprises adding the aqueous HA solution to the aqueous cation solution in a dropwise manner and the combining is carried out without stirring and the HA hydrogel is in bead form.

4. The method of claim 2, wherein the combining is carried out with simultaneous stirring, and the HA hydrogel is amorphous.

5. The method of claim 1, wherein the aqueous HA solution has a pH of about 11 to about 13.5, or the aqueous HA solution has a pH of about 12 to about 13.5.

6. The method of claim 1, wherein in (b) the method comprises adjusting the pH of the mixture to about 10 to about 13.5, and the combining of the aqueous HA solution and the aqueous cation solution or the solid divalent cation salt and the adjusting of the pH are carried out with simultaneous stirring, and wherein the HA hydrogel is amorphous.

7. The method of claim 6, wherein the adjusting of the pH of the mixture is carried out with a strong base, optionally the strong base is a hydroxide base, Tris base or mixtures thereof, optionally the strong base is an alkali hydroxide, optionally wherein the strong base is selected from NaOH, KOH, and combinations thereof, optionally, the strong base is an aqueous alkali hydroxide solution having a concentration of about 0.25 M to about 4 M.

8. The method of claim 7, wherein the strong base is present in the mixture at a concentration of about 10 mM to about 400 mM, about 15 mM to about 350 mM, about 15 mM to about 300 mM, or about 15 mM to about 280 mM.

9. The method of claim 8, wherein the mixture has a concentration of HA of about 0.1% w/v to about 4.5% w/v, about 0.1% w/v to about 4.0% w/v, about 0.1% w/v to about 3.5% w/v, about 0.1% w/v to about 3.0% w/v, about 0.125% w/v to about 2.5% w/v, about 0.25% w/v to about 2.5% w/v, about 0.75% w/v to about 2% w/v, about 0.5% w/v to about 2% w/v, or about 1% w/v.

10. The method of claim 1, wherein the divalent cation is present in the mixture at a concentration of about 3 mM to about 100 mM, about 10 mM to about 80 mM, about 15 mM to about 70 mM, about 20 mM to about 60 mM, about 25 mM to about 50 mM, about 25 mM to about 45 mM or about 30 mM.

11. The method of claim 1, wherein the divalent cation is Mg²⁺ or Zn²⁺.

12. The method of claim 1, wherein the divalent cation is Mg²⁺ and the concentration of the HA in the mixture is about 0.5% w/v to about 1.5% w/v, and wherein the HA and the Mg²⁺ are present in the mixture at a molar ratio of HA to Mg²⁺ of about 0.3 to about 2.5.

13. The method of claim 1, wherein the divalent cation is Zn²⁺ and the concentration of the HA in the mixture is about 0.5% w/v to about 1.5% w/v, and wherein the HA and the Zn²⁺ are present in the mixture at a molar ratio of HA to Zn²⁺ of about 0.3 to about 2.5.

14. The method of claim 1, wherein the divalent cation is in the form of its chloride salt, nitrate salt, acetate salt, sulfate salt and mixtures thereof, optionally the divalent cation is in the form of its chloride salt and/or nitrate salt.

15. The method of claim 1, wherein the HA in the aqueous HA solution is a hyaluronate salt, optionally sodium and/or potassium hyaluronate or is in acid form, or a mixture thereof.

16. The method of claim 1, wherein the HA has a molecular weight of about 200 kDa to about 1700 kDa, about 250 kDa to about 1600 kDa, about 300 kDa to about 1500 kDa, about 400 kDa to about 1400 kDa, about 500 kDa to about 1300 kDa, about 600 kDa to about 1200 kDa, about 700 kDa to about 1100 kDa, about 800 kDa to about 1000 kDa or about 800 kDa to about 900 kDa.

17. A hyaluronic acid (HA) hydrogel comprising divalent cations and HA molecules crosslinked non-covalently with the divalent cations, wherein the divalent cations are selected from Mg²⁺, Zn²⁺, Mn²⁺, Fe²⁺, Co²⁺, Pd²⁺, Ni²⁺, and combinations thereof.

18. A hyaluronic acid (HA) hydrogel prepared by the method of claim 1.

19. A biomaterial comprising the HA hydrogel of claim 18.

20. A method of delivering a drug to a subject in need thereof comprising administering a HA hydrogel of claim 18 to the subject.
